(19) United States
(12) Patent Application Publication
(10) Pub. No.: US 2004/0078851 A1
(43) Pub. Date: Apr. 22, 2004

(54) Production of human growth factors in monocot seeds

(76) Inventors: Ning Huang, Davis, CA (US); Daichang Yang, Sacramento, CA (US)

Correspondence Address: ARENT FOX KINTNER PLOTKIN & KAHN, 1050 CONNECTICUT AVENUE, N.W., SUITE 400, WASHINGTON, DC, 20036, US

(21) Appl. No.: 10/639,779

(22) Filed: Aug. 13, 2003

Related U.S. Application Data
(63) Continuation-in-part of application No. PCT/US02/04909, filed on Feb. 14, 2002.
(63) Continuation-in-part of application No. 09/847,232, filed on May 2, 2001.
(63) Continuation-in-part of application No. 10/077,381, filed on Feb. 14, 2002.
(63) Continuation-in-part of application No. 09/847,232, filed on May 2, 2001.
(60) Provisional application No. 60/269,199, filed on Feb. 14, 2001.
(60) Provisional application No. 60/269,188, filed on Feb. 14, 2001.
(60) Provisional application No. 60/266,929, filed on Feb. 6, 2001.
(60) Provisional application No. 60/201,182, filed on May 2, 2000.
(60) Provisional application No. 60/269,199, filed on Feb. 14, 2001.
(60) Provisional application No. 60/266,929, filed on Feb. 6, 2001.
(60) Provisional application No. 60/201,182, filed on May 2, 2000.

(51) Int. Cl. A01H 1/00; C12N 15/82; C07K 14/475

(52) U.S. Cl. 800/288; 435/468; 530/399

(57) ABSTRACT

Production of human growth factors in the seeds of monocot plants, vectors and transformed hosts for producing the same, and compositions comprising such growth factors and nucleic acids.

This application is a continuation-in-part of PCT/US02/04909, filed Feb. 14, 2002, which claims priority benefit to U.S. provisional application Serial No. 60/269,199 and U.S. provisional application Serial No. 60/269,188, each filed Feb. 14, 2001, PCT/US02/04909 being a continuation-in-part of U.S. patent application Ser. No. 09/847,232, filed May 2, 2001, which claims priority benefit to U.S. provisional application Serial No. 60/266,929, filed Feb. 6, 2001, and U.S. provisional application Serial No. 60/201,182, filed May 2, 2000. This application is also a continuation-in-part of U.S. patent application Ser. No. 10/077,381, filed Feb. 14, 2002, which claims priority benefit to U.S. provisional application Serial No. 60/269,199, filed Feb. 14, 2001, application Ser. No.10/077,381 being a continuation-in-part of U.S. patent application Ser. No. 09/847,232, filed May 2, 2001, which claims priority benefit to U.S. provisional application Serial No. 60/266,929, filed Feb. 6, 2001, and U.S. provisional application Serial No. 60/201,182, filed May 2, 2000. All priority applications are incorporated herein by reference in their entirety.

### FIELD OF THE INVENTION

The present invention relates to the production of human growth factors in the seeds of monocot plants, vectors and transformed hosts for producing the same, and compositions comprising such growth factors and nucleic acids.

### BACKGROUND OF THE INVENTION

Recombinant proteins have been expressed in vitro in different host expression systems such as bacterial cells, yeast and other fungi, mammalian cells, insect cells and, to a certain extent, plants. Each host expression system has its associated advantages and disadvantages.

Plants are attractive as hosts for expression of recombinant proteins as they are free from animal viruses and from toxins that are sometimes associated with microbial hosts. Scale-up can be performed more easily simply by planting more acres. Further, to the extent that the plant system is edible, recombinant molecules expressed in plant hosts may not require substantial purification if the recombinant molecules can retain bioactivity upon being ingested. Up to the present, the level of expression heterologous proteins in transgenic plants has been low and purification of recombinant proteins from portions of the plant, such as leaves, etc., can be costly, making such an expression system commercially impractical.

There is, thus, a need for a reliable method and system for effecting high level expression of recombinant or heterologous polypeptides in plants. Such a system may require a unique or novel combination of components parts such as one or more of: promoter, enhancer, transcription factor, codon-optimized heterologous gene, terminator, leader sequences, selectable marker, etc., that can operate efficiently together.

U.S. Pat. No. 5,994,628 discloses the production of proteins or polypeptides in the seeds of monocot plants such as rice. The promoters and signal sequences used for the process according to U.S. Pat. No. '628 include promoters and signal sequences from α-amylase genes, sucrose synthase genes or sucrose-6-phosphate synthetase genes that were expressed during the seed germination phase of plants development. Although growth factors are listed as one possible protein or polypeptide to be produced, no scientific data is provided concerning whether such production in germinating seeds was ever carried out.

Higo et al., Biosci. Biotech. Biochem. 57 (9), 1477-1481 (1993), discloses the production of human epidermal growth factor in tobacco plant leaves, by use of the cauliflower mosaic virus 35S promoter. Higo discloses that the highest yield of protein in tobacco leaf material was about 0.001% of total soluble protein.

There remains a need for a useful method of producing high levels of human growth factors in maturing monocot plant seeds. Growth factors produced via this method results in a non-animal based source of supply for the mammalian cell culture industry for production of therapeutic molecules.

### SUMMARY OF THE INVENTION

It is one of the objects of the present invention to address the unmet need for reliable methods for highlevel expression of human growth factors in the seeds of monocot plants.

It is another one of the objects of the present invention to provide vectors, hosts, and methods for such expression and compositions containing such.

Thus one embodiment of the present invention is a method of producing a human growth factor in monocot plant seeds, comprising the steps of:

(a) transforming a monocot plant cell with a chimeric gene comprising

(i) a promoter from a monocot plant gene that has upregulated activity during seed maturation,

(ii) a first DNA sequence, operably linked to said promoter, encoding a monocot plant seed-specific signal sequence capable of targeting a polypeptide linked thereto to monocot plant seed endosperm, and

(iii) a second DNA sequence, linked in translation frame with the first DNA sequence, encoding a human growth factor, wherein the first DNA sequence and the second DNA sequence together encode a fusion protein comprising an N-terminal signal sequence and the growth factor;

(b) growing a monocot plant from the transformed monocot plant cell for a time sufficient to produce seeds containing the growth factor; and

(c) harvesting the seeds from the plant.

Another embodiment of the present invention is a vector, comprising

(i) a promoter from a monocot plant gene that has upregulated activity during seed maturation,

(ii) a first DNA sequence, operably linked to said promoter, encoding a monocot plant seed-specific signal sequence capable of targeting a polypeptide linked thereto to monocot plant seed endosperm, and

(iii) a second DNA sequence, linked in translation frame with the first DNA sequence, encoding a human growth factor, wherein the first DNA sequence and the second DNA sequence together encode a fusion protein comprising an N-terminal signal sequence and the growth factor.

A further embodiment of the present invention is a transformed monocot plant cell, comprising

(i) a heterologous promoter from a monocot plant gene that has upregulated activity during seed maturation,

(ii) a first heterologous DNA sequence, operably linked to said promoter, encoding a monocot plant seed-specific signal sequence capable of targeting a polypeptide linked thereto to monocot plant seed endosperm, and

(iii) a second heterologous DNA sequence, linked in translation frame with the first DNA sequence, encoding a human growth factor, wherein the first DNA sequence and the second DNA sequence together encode a fusion protein comprising an N-terminal signal sequence and the growth factor.

Yet a further embodiment of the invention is a monocot plant seed product, such as whole seed, flour, extract, protein fraction or purified protein, prepared from the harvested seeds obtained according to the method of the invention. Preferably, the growth factor constitutes at least 0.1 weight percent of the total protein in the harvested seeds.

These and other objects and features of the invention will become more fully apparent when the following detailed description of the invention is read in conjunction with the accompanying figures.

### BRIEF DESCRIPTION OF THE FIGURES

FIG. 1 is a comparison of the codon-optimized epidermal growth factor sequence (“Egfactor”) (SEQ ID NO: 1, encoded protein shown in SEQ ID NO: 26) with a native epidermal growth factor sequence (“Native Gene”) (SEQ ID NO: 2) as disclosed in Bell et al. (1986) Nuc Acids Res 14: 8427-8446), aligned to show 53 codons in the mature sequences, with 27 (51%) codon changes and 30 (19%) nucleotide changes.

FIG. 2 is a restriction map of the 4,142 bp plasmid, pAPI270 (Glb-EGF-NOS), showing an expression cassette for epidermal growth factor (“EGF”), and containing a Glb promoter, a Glb signal peptide, codon optimized EGF, a Nos terminator and an ampicillin resistance selectable marker.

FIG. 3 is a restriction map of the 3,878 bp plasmid, pAPI303 (Gt1-EGF-NOS), showing an expression cassette for EGF, and containing a rice Gt1 promoter, a Gt1 signal peptide, codon optimized EGF, a Nos terminator and an ampicillin resistance selectable marker.

FIG. 4 is a Western blot analysis of recombinant human EGF (“rhEGF”) in the R1 generation of transgenic rice seeds. Lane 1 indicates extracts from seeds of control untransformed TP 309 rice variety; Lanes 2-5 show rhEGF expressed in the seed extracts obtained from independent transgenic rice events; Lane6 indicates a purified rhEGF standard expressed in yeast, loaded at 125 ng; Lane7 shows a broad range of molecular weight markers.

FIG. 5 is a comparison of the codon-optimized insulin-like growth factor I sequence (“Insgfact”) (SEQ ID NO: 3, encoded protein shown in SEQ ID NO: 27) with a native human insulin-like growth factor I sequence (“native gene”) (SEQ ID NO: 4) as disclosed in Rotwein, P., (1986). Proc Natl Acad Sci 83: 77 81, aligned to show 70 codons in the mature sequences, with 40 (57%) codon changes and 47 (22%) nucleotides changes.

FIG. 6 is a restriction map of the 4,193 bp plasmid, pAPI271 (Glb-IGF-NOS), showing an expression cassette for insulin-like growth factor I (“IGF”), and containing a Glb promoter, a Glb signal peptide, codon optimized IGF, a Nos terminator and an ampicillin resistance selectable marker.

FIG. 7 is a restriction map of the 3,928 bp plasmid, pAPI304 (Gt1-IGF-NOS), showing an expression cassette for insulin-like growth factor I (“IGF”), and containing a rice Gt1 promoter, a Gt1 signal peptide, codon optimized IGF, a Nos terminator and an ampicillin resistance selectable marker.

FIG. 8 is a Western blot analysis of recombinant human IGF-I (“rhIGF”) expressed in the R1 generation of transgenic rice seeds. Lane1 shows rice seed extract from seeds of control untransformed rice variety TP 309; Lanes 2-8 show rhIGF expressed in seed extracts obtained from seven independent transgenic rice events; Lane9 shows a purified rhIGF-1 standard expressed in yeast, loaded at 1 μg; Lane10 shows a broad range of molecular weight markers.

FIG. 9 is a comparison of the codon-optimized intestinal trefoil factor sequence (“Trefoil”) (SEQ ID NO: 5, encoded protein shown in SEQ ID NO: 28) with a native intestinal trefoil factor sequence (“Native Gene”) (SEQ ID NO: 6) as disclosed in (Podolsky et al., (1993). J Biol Chem 268: 6694-6702), aligned to show 60 codons in the mature sequences, with 26 (43%) codon changes and 28 (15%) nucleotide changes.

FIG. 10 is a restriction map of the 4,163 bp plasmid, pAPI269 (Glb-ITF-NOS), showing an expression cassette for intestinal trefoil factor (“ITF”), and containing a Glb promoter, a Glb signal peptide, codon optimized ITF, a Nos terminator and an ampicillin resistance selectable marker.

FIG. 11 is a restriction map of the 3,889 bp plasmid, pAPI307 (Gt1-ITF-NOS), showing an expression cassette for intestinal trefoil factor (ITF), and containing a rice Gt1 promoter, a Gt1 signal peptide, codon optimized ITF, a Nos terminator and an ampicillin resistance selectable marker.

FIG. 12 is a Western blot analysis of recombinant human ITF (“rhITF”) expression in the R1 generation of transgenic rice seeds. Lane1 indicates extracts from seeds of control untransformed TP 309 rice variety; Lanes2 and 3 show rhITF expressed in the seed extracts obtained from two independent transgenic rice events; Lane4 indicates a purified rhITF standard expressed in yeast, loaded at 1 μg; Lane5 shows a broad range of molecular weight markers.

### DETAILED DESCRIPTION OF THE INVENTION

Unless otherwise indicated, all terms used herein have the meanings given below or are generally consistent with same meaning that the terms have to those skilled in the art of the present invention. Practitioners are particularly directed to Sambrook et al. (1989) Molecular Cloning: A Laboratory Manual (Second Edition), Cold Spring Harbor Press, Plainview, N.Y., Ausubel F M et al. (1993) Current Protocols in Molecular Biology, John Wiley & Sons, New York, N.Y., and Gelvin and Schilperoot, eds. (1997) Plant Molecular Biology Manual, Kluwer Academic Publishers, The Netherlands, for definitions and terms of the art.

The polynucleotides of the invention may be in the form of RNA or in the form of DNA, and include messenger RNA, synthetic RNA and DNA, cDNA, and genomic DNA. The DNA may be double-stranded or single-stranded, and if single-stranded may be the coding strand or the non-coding (anti-sense, complementary) strand.

By “host cell” is meant a cell containing a vector and supporting the replication and/or transcription and/or expression of the heterologous nucleic acid sequence. Preferably, according to the invention, the host cell is a monocot plant cell. Other host cells may be used as secondary hosts, including bacterial, yeast, insect, amphibian or mammalian cells, to move DNA to a desired plant host cell.

A “plant cell” refers to any cell derived from a plant, including undifferentiated tissue (e.g., callus) as well as plant seeds, pollen, propagules, embryos, suspension cultures, meristematic regions, leaves, roots, shoots, gametophytes, sporophytes and microspores.

As used herein, the term “plant” includes reference to whole plants, plant tissues and individual plant cells, and progeny of same. Thus, the term includes, without limitation, leaves, stems, roots, shoots, endosperms, grains, seeds, embryos, suspension cultures, meristematic regions, callus tissue, gametophytes, sporophytes, pollen, progagules, and microspores. The class of plants includes higher plants amenable to transformation techniques, such as monocotyledenous and dicotyledenous plants.

The term “mature plant” refers to a fully differentiated plant.

As used herein, the term “seed” refers to all seed components, including, for example, the coleoptile and leaves, radicle and coleorhiza, scutulum, starchy endosperm, aleurone layer, pericarp and/or testa, either during seed maturation and seed germination. In the context of the present invention, the term “seed” and “grain” is used interchangeably.

The term “seed product” includes, but is not limited to, whole seed, seed fractions such as de-hulled whole seed, flour (seed that has been de-hulled by milling and ground into a powder), a seed extract, a protein fraction (where the protein portion of the seed has been separated from the carbohydrate portion), malt (including malt extract or malt syrup) and/or a purified protein derived from the seed or seed extract.

The term “biological activity” refers to any biological activity typically attributed to that protein by those of skill in the art.

The term “human growth factor” refers to proteins, or biologically active fragments thereof, including, without I imitation, epidermal growth factor (EGF), keratinocyte growth factors (KGF) including KGF-1 and KGF-2, insulin-like growth factors (IGF) including IGF-I and IGF-II, intestinal trefoil factor (ITF), transforming growth factors (TGF) including TGF-α and -β1-3, granulocyte colony-stimulating factor (GCSF), nerve growth factor (NGF) including NGF-β, and fibroblast growth factor (FGF) including FGF-1-19 and -12β, and biologically active fragments of these proteins. The sequences of these and other human growth factors are well-known to those of ordinary skill in the art.

The term “non-nutritional” refers to a pharmaceutically acceptable excipient which does not as its primary effect provide nutrition to the recipient. Preferably, it may provide one of the following services to an enterically delivered formulation, including acting as a carrier for a therapeutic protein, protecting the protein from acids in the digestive tract, providing a time-release of the active ingredients being delivered, or otherwise providing a useful quality to the formulation in order to administer to the patient the growth factors of the invention.

“Monocot seed components” refers to carbohydrate, protein, and lipid components extractable from monocot seeds, typically mature monocot seeds.

“Seed maturation” refers to the period starting with fertilization in which metabolizable reserves, e.g., sugars, oligosaccharides, starch, phenolics, amino acids, and proteins, are deposited, with and without vacuole targeting, to various tissues in the seed (grain), e.g., endosperm, testa, aleurone layer, and scutellar epithelium, leading to grain enlargement, grain filling, and ending with grain desiccation.

“Maturation-specific protein promoter” refers to a promoter exhibiting substantially upregulated activity (greater than 25%) during seed maturation.

“Heterologous DNA” refers to DNA which has been introduced into plant cells from another source, or which is from a plant source, including the same plant source, but which is under the control of a promoter that does not normally regulate expression of the heterologous DNA.

“Heterologous protein” is a protein encoded by a heterologous DNA.

A “signal sequence” is an N- or C-terminal polypeptide sequence which is effective to localize the peptide or protein to which it is attached to a selected intracellular or extracellular region, such as seed endosperm. Preferably, according to the invention, the signal sequence targets the attached peptide or protein to a location such as an endosperm cell, more preferably an endosperm-cell subcellular compartment or tissue, such as an intracellular vacuole or other protein storage body, chloroplast, mitochondria, or endoplasmic reticulum, or extracellular space, following secretion from the host cell.

As used herein, the terms “native” or “wild-type” relative to a given cell, polypeptide, nucleic acid, trait or phenotype, refers to the form in which that is typically found in nature.

As used herein, the term “purifying” is used interchangeably with the term “isolating” and generally refers to any separation of a particular component from other components of the environment in which it is found or produced. For example, purifying a recombinant protein from plant cells in which it was produced typically means subjecting transgenic protein-containing plant material to separation techniques such as sedimentation, centrifugation, filtration, column chromatography. The results of any of such purifying or isolating steps may still contain other components as long as the results have less other components (“contaminating components”) than before such purifying or isolating steps.

As used herein, the terms “transformed” or “transgenic” with reference to a host cell means the host cell contains a non-native or heterologous or introduced nucleic acid sequence that is absent from the native host cell. Further, “stably transformed” in the context of the present invention means that the introduced nucleic acid sequence is maintained through two or more generations of the host, which is preferably (but not necessarily) due to integration of the introduced sequence into the host genome.

The present invention provides for the production of human growth factors, or biologically active fragments thereof. Preferably, the growth factor constitutes at least 0.1 weight percent of the total protein in the harvested seeds. More preferably, the growth factor constitutes at least 0.25 weight percent of the total protein in the harvested seeds. In addition, the growth factor produced in the method optionally comprises one or more plant glycosyl groups. The plant glycosyl groups, while identifying that the growth factor was produced in a plant, does not significantly impair the biological activity of the growth factor in any of the applied therapeutic contexts (preferably less than 25% loss of activity, more preferably less than 10% loss of activity, as compared to a corresponding nonrecombinant growth factor). A purified growth factor recombinantly produced in a plant cell, preferably substantially free of contaminants of the host plant cell and free of any animal derived biological agents (viruses, prions, etc), is also provided by the invention.

In one embodiment of the invention, the nucleic acid sequence encoding the human growth factor is a native sequence. However, due to the inherent degeneracy of the genetic code, a number of nucleic acid sequences which encode substantially the same or a functionally equivalent amino acid sequence may be generated and used to clone and express a given growth factor, as exemplified herein by the codon optimized coding sequences used to practice the invention, and further described below. Thus, for a given growth factor-encoding nucleic acid sequence, it is appreciated that as a result of the degeneracy of the genetic code, a number of coding sequences can be produced that encode the same protein amino acid sequence. Such substitutions in the coding region fall within the range of sequence variants covered by the present invention. Any and all of these sequence variants can be utilized in the same way as described herein for the exemplified growth factor-encoding nucleic acid sequence.

A “variant” growth factor-encoding nucleic acid sequence may encode a “variant” growth factor amino acid sequence altered by one or more amino acids from the native sequence, both of which are included within the scope of the invention. Such variant sequences may contain at least one nucleic acid or amino acid substitution, deletion or insertion. The nucleic acid or amino acid substitution, insertion or deletion may occur at any residue within the sequence, as long as the encoded amino acid sequence maintains substantially the same (i.e., about 90% or greater) biological activity of the native sequence.

The variant nucleic acid coding sequence may encode a variant amino acid sequence which contains a “conservative” substitution, wherein the substituted amino acid has structural or chemical properties similar to the amino acid which it replaces and physicochemical amino acid side chain properties and high substitution frequencies in homologous proteins found in nature (as determined, e.g., by a standard Dayhoff frequency exchange matrix or BLOSUM matrix). In addition, or alternatively, the variant nucleic acid coding sequence may encode a variant amino acid sequence containing a “non-conservative” substitution, wherein the substituted amino acid has dissimilar structural or chemical properties to the amino acid which it replaces.

Standard substitution classes include six classes of amino acids based on common side chain properties and highest frequency of substitution in homologous proteins in nature, as is generally known to those of skill in the art and may be employed to develop variant growth factor-encoding nucleic acid sequences.

As will be understood by those of skill in the art, in some cases it may be advantageous to use a growth factor-encoding nucleotide sequences possessing non-naturally occurring codons. Codons preferred by a particular eukaryotic host can be selected, for example, to increase the rate of expression or to produce recombinant RNA transcripts having desirable properties, such as a longer half-life, than transcripts produced from naturally occurring sequence. As an example, it has been shown that codons for genes expressed in rice are rich in guanine (G) or cytosine (C) in the third codon position (Huang et al., J. CAASS 1: 73-86, 1990). Changing low G+C content to a high G+C content has been found to increase the expression levels of foreign protein genes in barley grains (Horvath et al., Proc. Natl. Acad. Sci. USA 97: 1914-19, 2000). The genes employed in the p resent invention may be based on the rice gene codon bias (Huang et al., supra) along with the appropriate restriction sites for gene cloning. These codon-optimized genes may be linked to regulatory and secretion sequences for seed-directed monocot expression and these chimeric genes then inserted into the appropriate plant transformation vectors. Codon-optimized sequences for use in practicing the invention are further described below.

The present invention provides for nucleic acid constructs, vectors, expression systems and methods for highlevel expression of human growth factors in monocot seeds, and compositions containing such as well as compositions resulting from such expression. For example, the monocots are cereals including rice, barley, wheat, oat, rye, corn, millet, triticale and sorghum.

One embodiment of the present invention is based on the expression of nucleic acid molecules encoding human growth factors which are each linked to a signal peptide for directing the expressed polypeptide to the protein bodies within an endosperm cell, under the control of one or more seed or maturation specific promoters, such as a promoter from a seed storage protein, or an aleurone- or embryo-specific promoter, with or without the addition of one or more transcription factors.

Expression vectors for use in the present invention are chimeric nucleic acid constructs (or expression vectors o r cassettes), designed for operation in plants, with associated upstream and downstream sequences.

In general, expression vectors for use in practicing the invention include the following operably linked components that constitute a chimeric gene: (i) a seed maturation-specific or an aleurone- or embryo-specific monocot plant gene promoter from a plant, (ii) operably linked to a leader DNA encoding a monocot seed-specific transit sequence capable of targeting a linked polypeptide to a seed of the plant, such as the leader sequence for targeting to a protein-storage body, and (iii) a heterologous human growth factor-encoding sequence.

The chimeric gene, in turn, is typically placed in a suitable plant-transformation (“expression”) vector having (i) companion sequences upstream and/or downstream of the chimeric gene which are of plasmid or viral origin and provide necessary characteristics to the vector to permit the vector to move DNA from one host to another, such as from bacteria to a desired plant host; (ii) a selectable marker sequence; and (iii) a transcriptional termination region with or without a polyA tail.

Exemplary methods for constructing chimeric genes and transformation vectors carrying the chimeric genes are given in the examples below.

In one aspect of this embodiment, the expression construct includes promoters from genes that exhibit substantially upregulated activity during seed maturation. Other examples of promoters useful according to the present invention include, but are not limited to the maturation-specific promoter associated with one of the following maturation-specific monocot storage proteins: rice glutelins, oryzins, and prolamines, barley hordeins, wheat gliadins and glutenins, maize zeins and glutelins, oat glutelins, and sorghum kafirins, millet pennisetins, rye secalins. Also included herein are aleurone and embryo specific promoters associated with the rice, wheat and barley genes such as lipid transfer protein Ltp1, chitinase Chi26 (Hwang et al., Plant Cell Rep. 20: 647-654 (2001)), and Em protein Emp1 (Litts et al., Plant Mol. Biol. 19: 335-337 (1992)). Exemplary regulatory regions from these genes are exemplified by SEQ ID NOS: 7-15.

In one embodiment of the present invention, a heterologous nucleic acid encoding a human growth factor is expressed under the control of a promoter from a transcription initiation region that is preferentially expressed in plant seed tissue. Examples of such seed preferential transcription initiation sequences include those derived from sequences encoding plant storage protein genes or from genes involved in fatty acid biosynthesis in oilseeds. Exemplary preferred promoters include a glutelin (Gt1) promoter, as exemplified in SEQ ID NO: 7, which effects gene expression in the outer layer of the endosperm and a globulin (Glb) promoter, as exemplified in SEQ ID NO: 8, which directs gene expression preferentially to the endosperm. Promoter sequences for regulating transcription of gene coding sequences operably linked thereto include naturally-occurring promoters, or regions thereof capable of directing seed-specific transcription, and hybrid promoters, which combine elements of more than one promoter. Methods for constructing such hybrid promoters are well known in the art.

In some cases, the promoter is derived from the same plant species as the plant cells into which the chimeric nucleic acid construct is to be introduced. Promoters for use in the invention are typically derived from cereals such as rice, barley, wheat, oat, rye, corn, millet, triticale or sorghum.

Alternatively, a seed-specific promoter from one type of plant may be used to regulate transcription of a nucleic acid coding sequence from a different plant.

Numerous types of appropriate expression vectors, and suitable regulatory sequences are known in the art for a variety of plant host cells. The transcription regulatory or promoter region is chosen to be regulated in a manner allowing for induction under seed-maturation conditions. Other promoters suitable for expression in maturing seeds include the barley endosperm-specific B1-hordein promoter (Brandt et al., Carlsberg Res. Commun. 50: 333-345 (1985)), GIuB-2 promoter, Bx7 promoter, Gt3 promoter, GIuB-1 promoter and Rp-6 promoter. Preferably, these promoters are used in conjunction with transcription factors.

In addition to encoding the protein of interest, the expression cassette or heterologous nucleic acid construct may encode a signal peptide that allows processing and translocation of the protein, as appropriate. Exemplary signal sequences, particularly for targeting proteins to intracellular bodies, such as vacuoles, are signal sequences associated with the monocot maturation-specific genes: glutelins, prolamines, hordeins, gliadins, glutenins, zeins, albumin, globulin, ADP glucose pyrophosphorylase, starch synthase, branching enzyme, Em, and lea. Exemplary sequences encoding a leader sequence for protein storage body are identified herein as SEQ ID NOS: 16-22.

In one embodiment of the present invention, the method is directed toward the localization of heterologous polypeptide expression in a given subcellular compartment or tissue, such as protein-storage body, aleurone layers or embryo, but also including other compartments such as vacuoles, chloroplasts or other plastidic compartments or mitochondria. For example, when heterologous polypeptide expressed is targeted to plastids, such as chloroplasts, the construct employs the use of sequences to direct the gene to the plastid. Such sequences are for example chloroplast transit peptides (CTP) or plastid transit peptides (PTP). In this manner, when the gene of interest is not directly inserted into the plastid, the expression construct additionally contains a gene encoding a transit peptide to direct the gene of interest to the plastid. The chloroplast transit peptides may be derived from the gene of interest, or may be derived from a heterologous sequence having a CTP. Such transit peptides are known in the art. See, for example, Von Heijne et al., Plant Mol. Biol. Rep. 9:104-126, 1991; Clark et al., J. Biol. Chem. 264:17544-17550, 1989; dellaCioppa et al., Plant Physiol. 84:965-968, 1987; Romer et al., Biochem. Biophys. Res Commun. 196:1414-1421, 1993; and Shah et al., Science 233:478-481, 1986. Additional transit peptides for the translocation of the protein to the endoplasmic reticulum (ER) (Chrispeels, Ann. Rev. Plant Phys. Plant Mol. Biol. 42:21-53, 1991), nuclear localization signals (Raikhel, Plant Phys. 100:1627, 1632, 1992), or vacuole may also find use in the constructs of the present invention.

Another exemplary class of signal sequences are sequences effective to promote secretion of heterologous protein from aleurone cells during seed germination, including the signal sequences associated with α-amylase, protease, carboxypeptidase, endoprotease, ribonuclease, DNase/RNase, (1-3)β-glucanase, (1-3)(1-4)-β-glucanase, esterase, acid phosphatase, pentosamine, endoxylanase, β-xylopyranosidase, arabinofu ranosidase, β-glucosidase, (1-6)-βglucanase, perioxidase, and lysophospholipase.

Since many protein storage proteins are under the control of a maturation-specific promoter, and this promoter is operably linked to a leader sequence for targeting to a protein body, the promoter and leader sequence can be isolated from a single protein-storage gene, then operably linked to a heterologous polypeptide in a chimeric gene construct. One exemplary promoter-leader sequence is from the rice Gt1 gene, having an exemplary sequence identified in SEQ ID NO: 7. Alternatively, the promoter and leader sequence may be derived from different genes. Another exemplary promoter/leader sequence combination is the rice Glb promoter linked to the rice Gt1 leader sequence, as exemplified by SEQ ID NO: 8.

It has been shown that production of recombinant protein in transgenic barley grain was enhanced by codon optimization of the gene (Horvath et al., Proc. Natl. Acad. Sci. USA, 97:1914-1919, 2000; Jensen et al., Proc. Natl. Acad. Sci. USA, 93:3487-3491, 1996). The intent of codon optimization was to change an A or T at the third position of the codons of G or C. This arrangement conforms more closely with codon usage in typical rice genes (Huang et al., J CAASS, 1:73-86, 1990). Such codon optimization is intended to be within the scope of the present invention.

In one embodiment of the invention, the transgenic plant herein is also transformed with the coding sequence of one or more transcription factors capable of enhancing the expression of a maturation-specific promoter. For example, one embodiment involves the use of the maize Opaque 2 (O2) or prolamin box binding factor (PBF), separately or together, or the use of rice endosperm b Zip ( Reb) protein as transcriptional activators herein. Exemplary sequence for these three transcription factors are given identified below as SEQ ID NOS: 23-25. Transcription factor sequences and constructs applicable to the present invention are detailed in WO 01/83792.

Transcription factors are capable of sequence-specific interaction with a gene sequence or gene regulatory sequence. The interaction may be direct sequence-specific binding in that the transcription factor directly contacts the gene or gene regulatory sequence or indirect sequence-specific binding mediated by interaction of the transcription factor with other proteins. In some cases, the binding and/or effect of a transcription factor is influenced (in an additive, synergistic or inhibitory manner) by another transcription factor. The gene or gene regulatory region and transcription factor may be derived from the same type (e.g., species or genus) of plant or a different type of plant. The binding of a transcription factor to a gene sequence or gene regulatory sequence may be evaluated by a number of assays routinely employed by those of skill in the art, for example, sequence-specific binding may be evaluated directly using a label or through gel shift analysis.

As detailed in the cited WO publication, the transcription factor gene is introduced into the plant in a chimeric gene containing a suitable promoter, preferably a maturation-specific seed promoter operably linked to the transcription factor gene. Plants may be stably transformed with a chimeric gene containing the transcription factor by methods similar to those described with respect to the growth factor genes exemplified herein. Plants stably transformed with both exogenous transcription factors and growth factor genes may be prepared by co-transforming plant cells or tissue with both gene constructs, selecting plant cells or tissue that have been co-transformed, and regenerating the transformed cells or tissue into plants. Alternatively, different plants may be separately transformed with exogenous transcription factor genes and growth factor genes, then crossed to produce plant hybrids containing the added genes.

Expression vectors or heterologous nucleic acid constructs designed for operation in plants may comprise companion sequences upstream and downstream to the expression cassette. The companion sequences are of plasmid or viral origin and provide necessary characteristics to the vector to permit the vector to move DNA from one host to another such as from bacteria to the plant host including, for example, sequences containing an origin of replication and a selectable marker. Typical secondary hosts for production of plasmids for transformation into plants include bacteria and yeast.

In one embodiment, the secondary host is E. coli, the origin of replication is a colE1-type, and the selectable marker is a gene encoding ampicillin resistance. Such sequences are well known in the art and are commercially available as well (e.g., Clontech, Palo Alto, Calif.; Stratagene, La. Jolla, Calif.).

The transcription termination region may be taken from a gene where it is normally associated with the transcriptional initiation region or may be taken from a different gene. Exemplary transcriptional termination regions include the NOS terminator from Agrobacterium Ti plasmid and the rice α-amylase terminator.

Polyadenylation tails (Alber and Kawasaki, Mol. and Appl. Genet. 1:419-434, 1982) may also be added to the expression cassette to optimize high levels of transcription and proper transcription termination, respectively. Polyadenylation sequences include, but are not limited to, the Agrobacterium octopine synthetase signal (Gielen et al., EMBO J. 3:835-846, 1984) or the nopaline synthase of the same species (Depicker et al., Mol. Appl. Genet. 1:561 573,1982).

Suitable selectable markers for selection in plant cells include, but are not limited to, antibiotic resistance genes, such as kanamycin (nptII), G418, bleomycin, hygromycin, chloramphenicol, ampicillin, tetracycline, and the like. Additional selectable markers include a bar gene which codes for bialaphos resistance; a mutant EPSP synthase gene which encodes glyphosate resistance; a nitrilase gene which confers resistance to bromoxynil; a mutant acetolactate synthase gene (ALS) which confers imidazolinone or sulphonylurea resistance; and a methotrexate resistant DHFR gene.

The particular marker gene employed is one which allows for selection of transformed cells as compared to cells lacking the DNA which has been introduced. Preferably, the selectable marker gene is one which facilitates selection at the tissue culture stage, e.g., a kanamyacin, hygromycin or ampicillin resistance gene.

The vectors of the present invention may also be modified to include intermediate plant transformation plasmids that contain a region of homology to an Agrobacterium tumefaciens vector, a T-DNA border region from Agrobacterium tumefaciens, and chimeric genes or expression cassettes (described above). Further, the vectors of the invention may comprise a disarmed plant tumor inducing plasmid of Agrobacterium tumefaciens.

In general, a selected nucleic acid sequence is inserted into an appropriate restriction endonuclease site or sites in the vector. Standard methods for cutting, ligating and E. coli transformation, known to those of skill in the art, are used in constructing vectors for use in the present invention. (See generally, Maniatis et al., MOLECULAR CLONING: A LABORATORY MANUAL, 2d Edition (1989); Ausubel et al., (c) 1987, 1988, 1989, 1990, 1993, CURRENT PROTOCOLS IN MOLECULAR BIOLOGY, John Wiley & Sons, New York, NY; and Gelvin et al., eds. PLANT MOLECULAR BIOLOGY MANUAL (1990).

Plant cells or tissues are transformed with expression constructs (heterologous nucleic acid constructs), for example, plasmid DNA, into which the gene of interest has been inserted) using a variety of standard techniques. It is preferred that the vector sequences be stably integrated into the host genome.

The method used for transformation of host plant cells is not critical to the present invention. For commercialization of recombinant growth factors expressed in accordance with the present invention, the transformation of the plant is preferably permanent, i.e. by integration of the introduced expression constructs into the host plant genome, so that the introduced constructs are passed onto successive plant generations. The skilled artisan will recognize that a wide variety of transformation techniques exist in the art, and new techniques are continually becoming available.

Any technique that is suitable for the target host plant may be employed within the scope of the present invention. For example, the constructs can be introduced in a variety of forms including, but not limited to, as a strand of DNA, in a plasmid, or in an artificial chromosome. The introduction of the constructs into the target plant cells can be accomplished by a variety of techniques, including, but not limited to calcium-phosphate-DNA co-precipitation, electroporation, microinjection, Agrobacterium-mediated transformation, liposome-mediated transformation, protoplast fusion or microprojectile bombardment (Christou, P. (1992). Plant Jour 2: 275-281). The skilled artisan can refer to the literature for details and select suitable techniques for use in the methods of the present invention.

When Agrobacterium is used for plant cell transformation, a vector is introduced into the Agrobacterium host for homologous recombination with TDNA or the Ti- or Ri-plasmid present in the Agrobacterium host. The Ti- or Riplasmid containing the T-DNA for recombination may be armed (capable of causing gall formation) or disarmed (incapable of causing gall formation), the latter being permissible, so long as the vir genes are present in the transformed Agrobacterium host. The armed plasmid can give a mixture of normal plant cells and gall.

In some instances where Agrobacterium is used as the vehicle for transforming host plant cells, the expression or transcription construct bordered by the T-DNA border region(s) is inserted into a broad host range vector capable of replication in E. coli and Agrobacterium, examples of which are described in the literature, for example pRK2 or derivatives thereof. See, for example, Ditta et al., Proc. Nat. Acad. Sci., U.S.A. 77:7347-7351, 1980 and EP 0 120 515. Alternatively, one may insert the sequences to be expressed in plant cells into a vector containing separate replication sequences, one of which stabilizes the vector in E. coli, and the other in Agrobacterium See, for example, McBride and Summerfelt, Plant Mol. Biol. 14:269-276, 1990, wherein the pRiHRI (Jouanin et al., Mol. Gen. Genet. 201:370-374, 1985) origin of replication is utilized and provides for added stability of the plant expression vectors in host Agrobacterium cells.

Included with the expression construct and the T-DNA is one or more selectable marker coding sequences which allow for selection of transformed Agrobacterium and transformed plant cells. A number of markers have been developed for use with plant cells, such as resistance to chloramphenicol, kanamycin, the aminoglycoside G418, hygromycin, or the like. The particular marker employed is not essential to this invention, with a particular marker preferred depending on the particular host and the manner of construction.

For Agrobacterium-mediated transformation of plant cells, explants are incubated with Agrobacterium for a time sufficient to result in infection, the bacteria killed, and the plant cells cultured in an appropriate selection medium. Once callus forms, shoot formation can be encouraged by employing the appropriate plant factors in accordance with known methods and the shoots transferred to rooting medium for regeneration of plants. The plants may then be grown to seed and the seed used to establish repetitive generations and for isolation of the recombinant protein produced by the plants.

There are a number of possible ways to obtain plant cells containing more than one expression construct. In one approach, plant cells are co-transformed with a first and second construct by inclusion of both expression constructs in a single transformation vector or by using separate vectors, one of which expresses desired genes. The second construct can be introduced into a plant that has already been transformed with the first expression construct, or alternatively, transformed plants, one having the first construct and one having the second construct, can be crossed to bring the constructs together in the same plant.

Transformed plant cells are screened for the ability to be cultured in selective media having a threshold concentration of a selective agent. Plant cells that grow on or in the selective media are typically transferred to a fresh supply of the same media and cultured again. The explants are then cultured under regeneration conditions to produce regenerated plant shoots. After shoots form, the shoots are transferred to a selective rooting medium to provide a complete plantlet. The plantlet may then be grown to provide seed, cuttings, or the like for propagating the transformed plants. The method provides for efficient transformation of plant cells with expression of a gene of autologous or heterologous origin and regeneration of transgenic plants, which can produce a recombinant growth factor.

The expression of the recombinant growth factor may be confirmed using standard analytical techniques such as Western blot, ELISA, PCR, HPLC, NMR, or mass spectroscopy, together with assays for a biological activity specific to the particular protein being expressed.

The invention provides, in one aspect, a plant seed product prepared from the harvested seeds obtained by the method. The plant seed product is preferably composed of whole seed, seed fraction, flour, extract, malt, protein fraction or purified protein. Optionally, the plant seed product may contain a vehicle in a form suitable for human or animal use. For use in a food or feed product, the vehicle may be a capsule, binder components effective to tabletize the composition, a consumable liquid, or a consumable suspension. The vehicle may be a processed food in which the product is mixed. Below are described methods for preparing flour, extract, or malt compositions.

The flour composition is prepared by milling mature monocot plant seeds, using standard milling and, optionally, flour purification methods, e.g., in preparing refined flour. Briefly, mature seeds are dehusked, and the dehusked seeds then ground into a fine flour by conventional milling equipment.

The flour may be added to foods during food processing according to standard food processing methods. Preferably, the processing temperature does not lead to denaturation of the growth factors, e.g., the temperature does not rise above 70° C. The flour may also be used directly, either in capsule, tabletized, or powder form, as a nutraceutical composition. For producing cosmetic or care products, such as topical creams, the flour may be blended with vehicles suitable for this purpose. For preparing a surgical dressing or surgical powder, the vehicle is a surgical dressing or container for delivering the powder.

An extract composition may be prepared by milling seeds to form a flour, extracting the flour with an aqueous buffered solution, and optionally, further treating the extract to partially concentrate the extract and/or remove unwanted components. I n one embodiment, mature monocot seeds, such as rice seeds, are milled to a flour, and the flour then suspended in saline or in a buffer, such as Phosphate Buffered Saline (“PBS”), ammonium bicarbonate buffer, ammonium acetate buffer, Tris buffer or a volatile buffer that would evaporate upon drying. The flour suspension may be incubated with shaking for a period typically between 30 minutes and 4 hours, at a temperature between 20-55° C. The resulting homogenate may be clarified either by filtration or centrifugation. The clarified filtrate or supernatant may be further processed, for example by ultrafiltration or dialysis or both to remove contaminants such as lipids, sugars and salt. Finally, the material may be dried, e.g., by lyophilization, to form a dry cake or powder. The extract has the advantage of high recombinant polypeptide yields, limiting losses associated with protein purification. At the same time, the recombinant growth factors are in a form readily usable and available upon ingestion of the extract or food containing the extract.

One particular advantage of the extract is the low amount of seed starch present in the extract. In particular, the extract may increase the concentration of recombinant protein, from a lower limit of about 0.5% of total soluble protein (“TSP”) in the seed to about 25% or more of TSP in the extract. Concentrations of above 40% of TSP are possible depending on the expression level of the recombinant protein in the seeds. In addition, the extract approach removes starch granules, which require high gelling temperature, for example above about 75° C. Consequently, the extract approach provides more flexibility in processing the seeds.

The extract can be used in ways similar to the flour described above, and similar vehicles may be employed for delivering the proteins contained in the extract.

In accordance with another embodiment, the invention provides a malt extract or malt syrup (“malt”) composition in which seed starches have been largely reduced to malt sugars, and the growth factors are in an active, bioavailable form. The procedure for producing a malt is well-known, and is summarized in WO 02/064750.

The present invention also provides compositions comprising human growth factors produced recombinantly in the seeds of monocot plants, and methods of making such compositions. In practicing the invention, a human growth factor is produced in the seeds of transgenic plants that express the nucleic acid coding sequence for the growth factor. After expression, the growth factor may be provided to a patient in substantially unpurified form (i.e., at least 20% of the composition comprises plant material), or the growth factor may be isolated or purified from the plant seed product and formulated for delivery to a patient. Such compositions can comprise a formulation for the type of delivery intended. Delivery types can include, e.g. parenteral, enteric, inhalation, intranasal or topical delivery. Parenteral delivery can include, e.g. intravenous, intramuscular, or suppository. Enteric delivery can include, e.g. oral administration of a pill, capsule, or other formulation made with a non-nutritional pharmaceutically-acceptable excipient, or a composition with a nutrient from the transgenic plant, for example, in the extract in which the protein is made, or from a source other than the transgenic plant. Such nutrients include, for example, salts, saccharides, vitamins, minerals, amino acids, peptides, and proteins other than the growth factor. Intranasal and inhalant delivery systems can include spray or aerosol in the nostrils or mouth. Topical delivery can include, e.g. creams, topical sprays, or salves. Preferably, the composition is substantially free of contaminants of the transgenic plant, preferably containing less than 20% plant material, more preferably less than 10%, and most preferably, less than 5%. Preferably the excipient is non-nutrititional.

The following examples illustrate but are not intended in any way to limit the invention.

### EXAMPLE 1

In general, expression vectors were constructed using standard molecular biological techniques as described in Ausubel et al., 1987. The vectors contain a heterologous protein coding sequence for certain growth factors under the control of a rice tissue-specific promoter, as further described below.

The nucleotide sequence of the promoter and the nucleotide sequence of the signal peptide of the rice glutelin-1 (Gt1) gene were cloned based on the published Gt1 gene sequence (Okita et al. J. Biol. Chem. 264: 12573-12581, 1989). The nucleotide sequence of the promoter and the nucleotide sequence of the signal peptide of the rice globulin (Glb) gene were cloned based on the published Glb gene sequence (Nakase et al, (1996). Gene 170: 223-226).

A. Generation of Human EGF

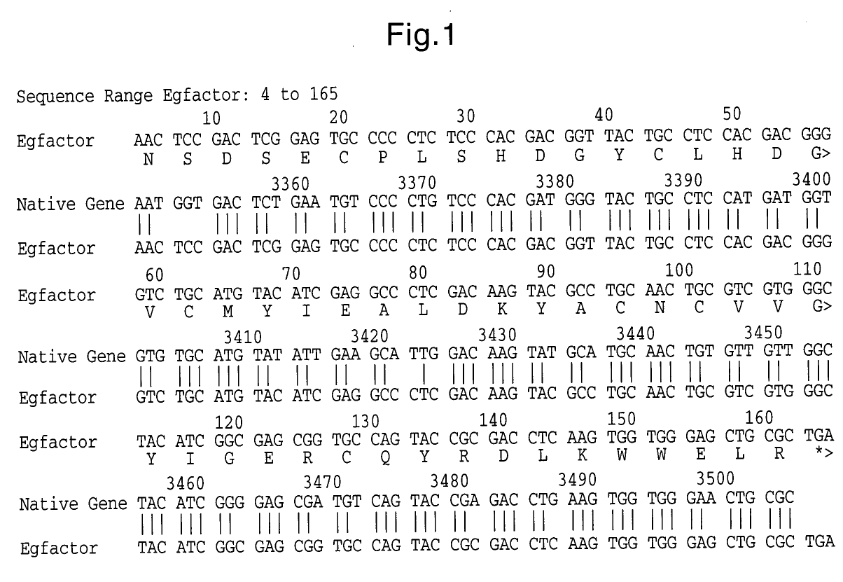

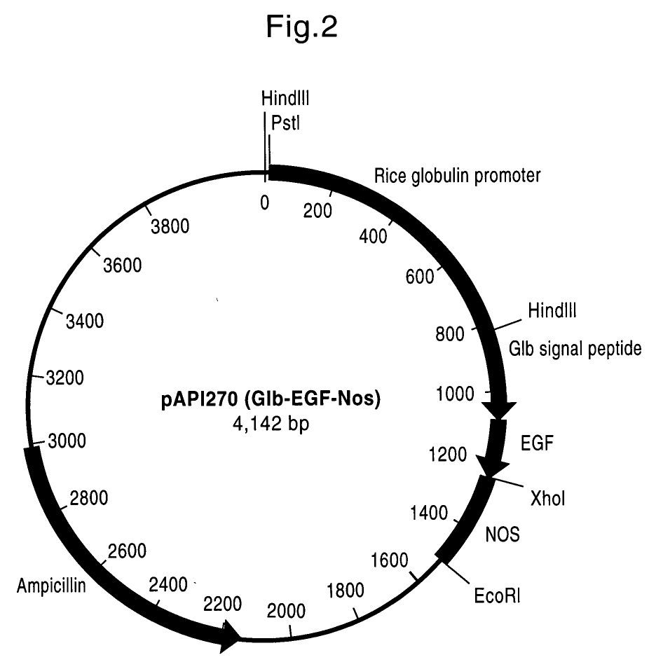

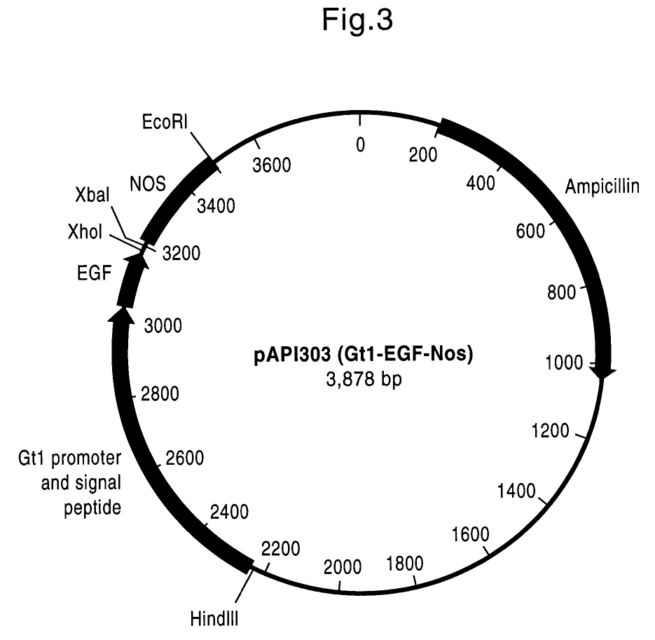

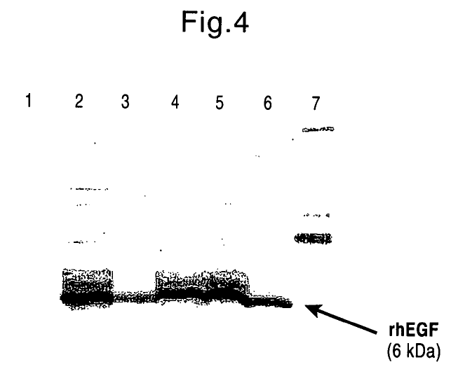

The human EGF gene was codon optimized as shown in FIG. 1, and synthesized by Operon Technologies (Calif., U.S.A.) (SEQ ID NO: 1). For expression of EGF in rice seeds, the codon optimized gene was operably linked to the rice endosperm specific glutelin (Gt1) promoter, Gt1 signal peptide and NOS terminator in pAPI303 (FIG. 3), and to the rice endosperm specific globulin (Glb) promoter, Glb signal peptide and NOS terminator in pAPI270 (FIG. 2). The transgenic plant expressing EGF was generated, and plant-generated recombinant EGF was detected, as shown in FIG. 4 and as exemplified herein.

B. Generation of Human IGF-I

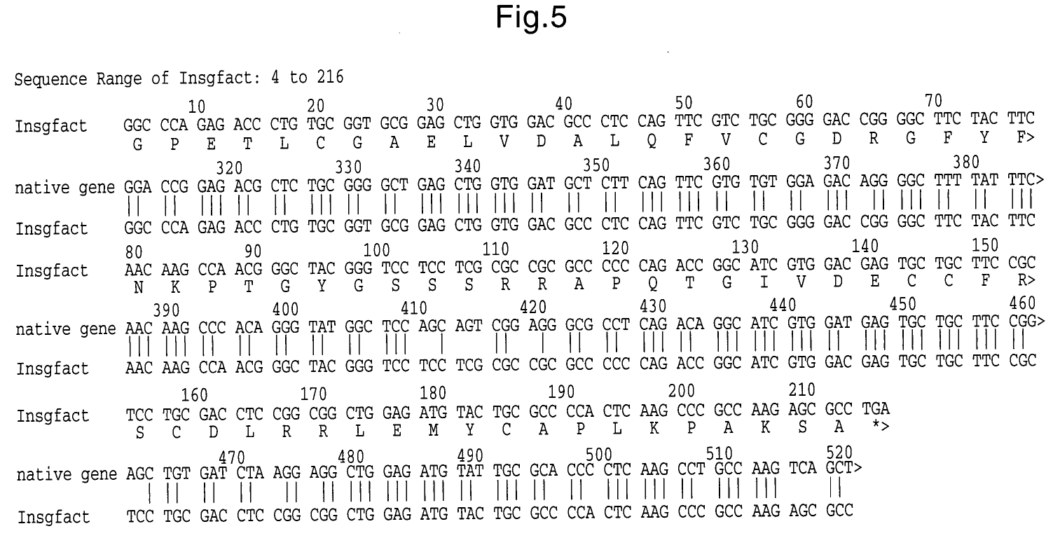

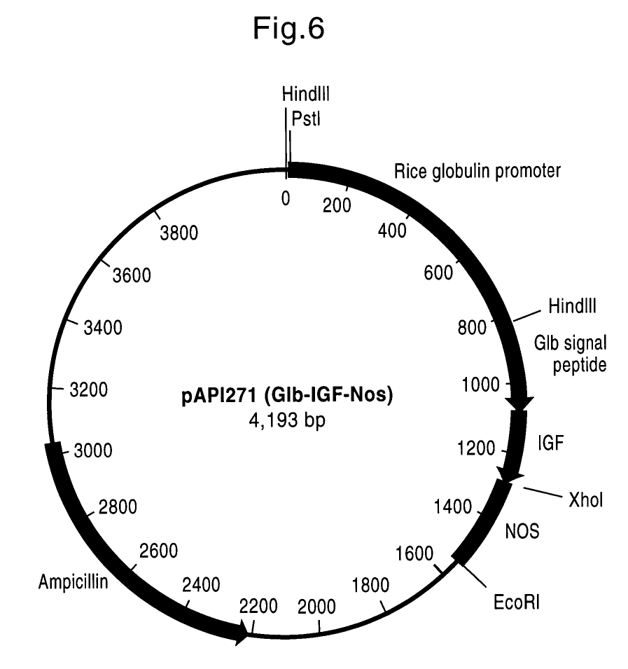

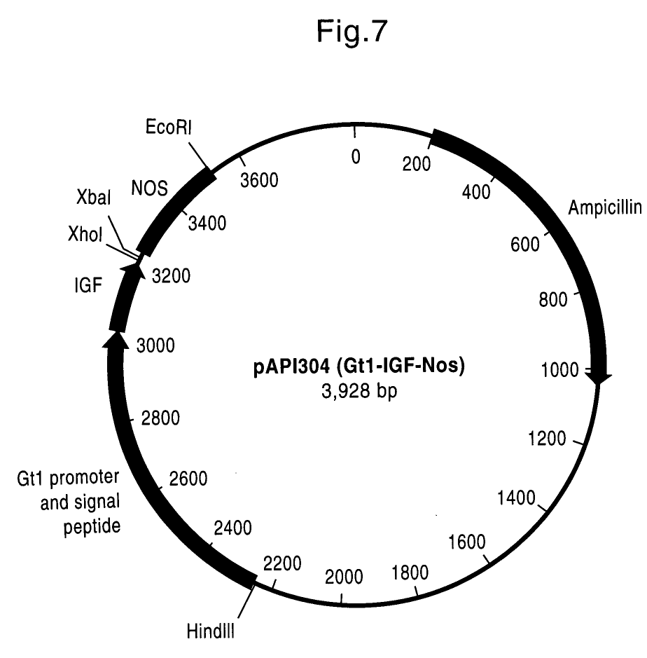

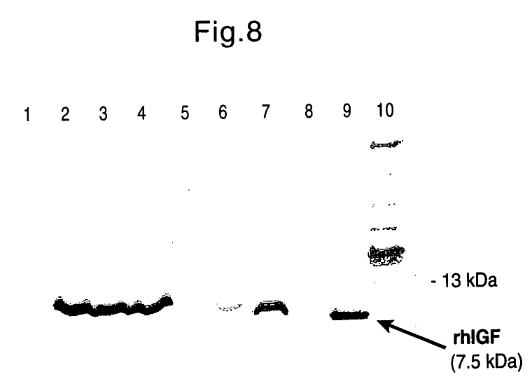

The IGF-I gene was codon optimized as shown in FIG. 5, and synthesized by Operon Technologies (Calif., USA) (SEQ ID NO: 3). For expression of IGF-I in rice seeds, the codon optimized gene was operably linked to the rice endosperm specific glutelin (Gt1) promoter, Gt1 signal peptide and NOS terminator in pAPI304 (FIG. 7), and to the rice endosperm specific globulin (Glb) promoter, Glb signal peptide and NOS terminator in pAPI271 (FIG. 6). The transgenic plant expressing IGF-I was generated, and plantgenerated recombinant IGF-I was detected as shown in FIG. 8 and as exemplified herein.

C. Generation of Human ITF

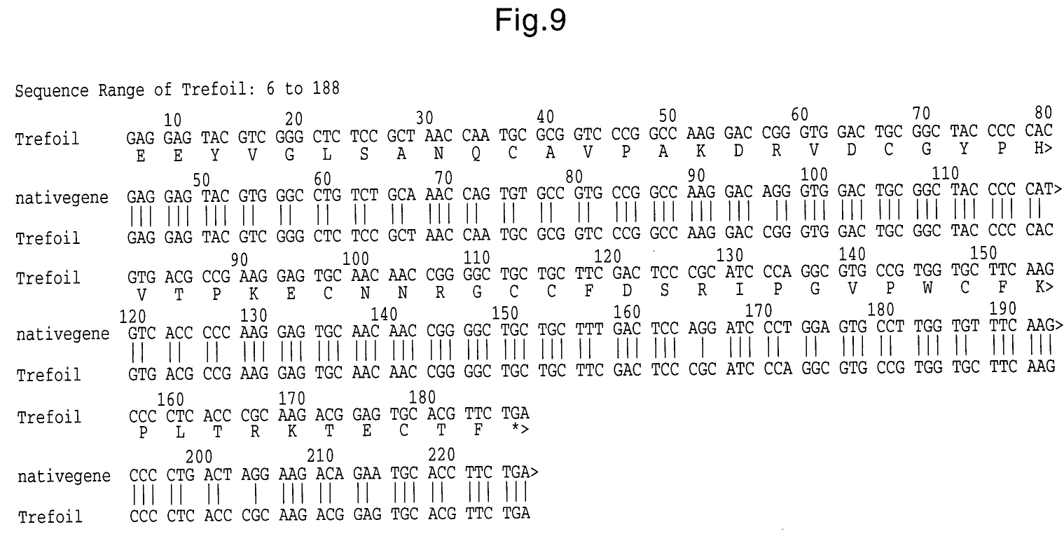

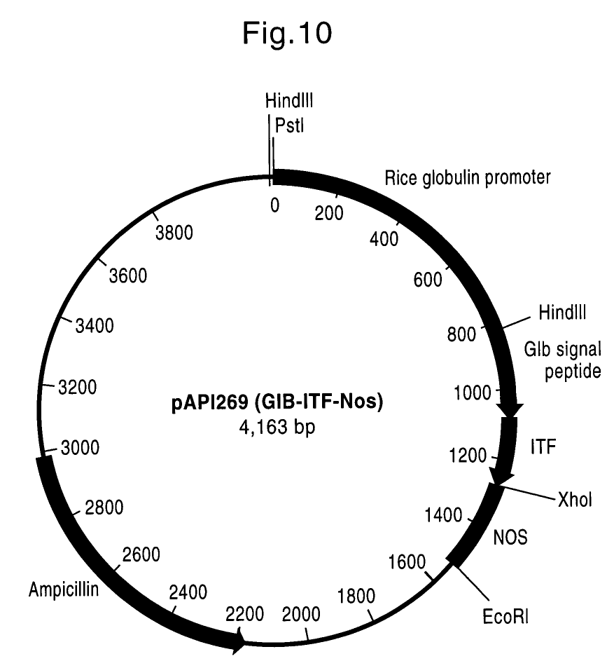

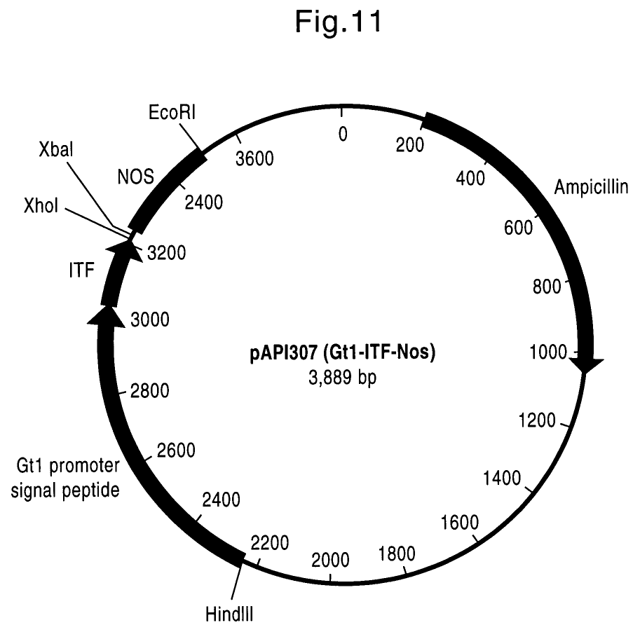

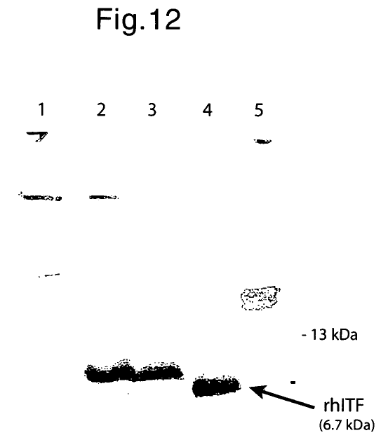

The ITF gene was codon optimized as shown in FIG. 9, and synthesized by Operon Technologies (Calif., USA) (SEQ ID NO: 5). For expression of ITF in rice seeds, the codon optimized gene was operably linked to the rice endosperm specific glutelin (Gt1) promoter, Gt1 signal peptide and NOS terminator in pAPI307 (FIG. 11), and to the rice endosperm specific globulin (Glb) promoter, Glb signal peptide and NOS terminator in pAPI269 (FIG. 10). The transgenic plant expressing ITF was generated, and plant-generated recombinant ITF was detected as shown in FIG. 12 and as exemplified herein.

### EXAMPLE 2

Western Blot Analysis for all Growth Factors

Both untransformed (rice var. Taipei 309) and transgenic rice seeds (˜10 pooled R1 seed from individual transgenic plants expressing either EGF, IGF-I or ITF) were ground in 1 ml of 0.35 M NaCl in phosphate buffered saline (PBS), pH 7.4, using an ice-cold mortar and pestle. The resulting extract was spun at 14,000 rpm at 4° C. for 10 min. Cleared supernatant was collected and ˜20 mg of this soluble protein extract was resuspended in sample loading buffer, and loaded onto a precast 10-20% polyacrylamide tricine gel (Novex) and subjected to SDS-PAGE. After electrophoresis, the gel was electroblotted to a 0.45 μm nitrocellulose membrane. The blot was blocked with 5% non-fat dry milk in PBS pH 7.4 for 2 hrs followed by three washes with PBS for 10 min each. A primary rabbit polyclonal antibody prepared against EGF(Sigma), IGF-I (Sigma) or ITF (GI Company) was used at 1:2000 dilution in PBS. Bands were developed using goat anti-rabbit antibody coupled to the BCIP/NBT substrate system (Sigma).

Results are shown in FIGS. 4, 8 and 12, respectively.

All references cited supra are expressly incorporated herein by reference. In addition, the following references are incorporated herein by reference to the extent they may be pertinent to the practice of the invention.

Arnold R. R. et al., Infect Immun. 28:893-898, 1980.

Bhan, M. K. et al., J Pediatr Gastroenterol Nutr 7:208-213, 1988.

Boesman-Finkelstein M. and Finkelstein R. A. FEBS Letters, 144:1-5, 1982.

Bradford M. Analytical Biochem. 72:248-254, 1976.

Bullen J. J. et al., Br. Med. J. 1, 69-75, 1972.

Castañon M. J. et al.,. Gene, 66:223-234,1988.

Chandan R. C., J Dairy Sci, 51:606-607,1968.

Chong, D. K. and Langridge, W. H., Transgenic Res 9:71-8, 2000.

Dellaporta S. L. et al., Plant Mol. Biol. Rep. 1:19-21, 1983.

Dewey K. G. et al., J Pediatrics 126:696-702, 1995.

Dewey K. G. et al., Pediatrics 89:1035-1041, 1992.

Dewey K. G. et al., Am J Clin Nutr 57:140-145, 1993.

Faure A. and Jollès P., et al., Comptes Rendus Hebdomadaires des Seances de L Academie des Sciences. D: Sciences Naturelles, 271:1916-1918, 1970.

Fujihara T. and Hayashi K., Archives of Virology 140:1469-1472, 1995.

Gastanaduy, A. et al., J Pediatr Gastroenterol Nutr 11:240-6,1990.

Gelvin, S. B. etal., eds. PLANT MOLECULAR BIOLOGY MANUAL, 1990.

Grover M. et al., Acta Paediatrica 86:315-316, 1997.

Harmsen M. C. et al., Journal of Infectious Diseases 172:380-388, 1995.

Huang N. et al., Plant Mol. Biol., 23:737-747, 1993.

Jigami Y. et al., Gene, 43:273-279,1986.

Kovar M. G. et al., Pediatrics 74:615-638,1984.

Kunz C. et al., Clin. Perinatol. 26(2):307-33, 1999.

Langridge et al., Planta 156:166-170, 1982.

Lee-Huang S. et al., Proc. Natl. Acad. Sci. USA, 96:2678-2681, 1999.

Lönnerdal B., Am L Clin Nutr, 42:1299-1317, 1985.

Maga E. etal., J. of Food Protection, 61:52-56, 1998.

Maga E. et aL, Transgenic Research, 3:36-42, 1994.

Maga E. et al., Journal of Dairy Science, 78:2645-2652, 1995.

Matsumoto, A. et al., Plant Mol Bio 27:1163-72, 1995.

Mitra A. and Zhang Z., Plant Physiol. 106:977-981, 1994.

Mitra, A. and Zhang, Z., Plant Physiol 106:977-81, 1994.

Motil K. J., Curr Opin Pediatr 12(5):469-76, 2000.

Nakajima H. et al., Plant Cell Reports, 16:674-679, 1997.

Rodriguez et al., U.S. Pat. No. 6,284,956.

Saarinen K. M. et al., Adv Exp Med Biol478:121-30, 2000.

Salmon, V. et al., Protein Expr Purif 13:127-135, 1998.

Samaranayake Y. H. et al., Apmis, 105:875-883, 1997.

Takai I. et a., J. Chrom. B, Biomedical Applications, 685:21-25, 1996.

Tsuchiya K. et al., Applied Microbiology and Biotechnology, 38:109-114, 1992.

Wang C. S. and Kloer H. U., Anal. Biochem., 139:224-227,1984.

Wang C. et al., Comp. Biochem. Physiol. 78B:575-580, 1984.

Ward P. P. et al., Bio/technology 10:784-789, 1992.

Yoshimura K. et al., Biochem. Biophys Res Com, 150:794-801, 1988.

What is claimed is:

1. A method of producing a human growth factor in monocot plant seeds, comprising the steps of:
(a) transforming a monocot plant cell with a chimeric gene comprising
(i) a promoter from a monocot plant gene that has upregulated activity during seed maturation,
(ii) a first DNA sequence, operably linked to said promoter, encoding a monocot plant seed-specific signal sequence capable of targeting a polypeptide linked thereto to monocot plant seed endosperm, and
(iii) a second DNA sequence, linked in translation frame with the first DNA sequence, encoding a human growth factor, wherein the first DNA sequence and the second DNA sequence together encode a fusion protein comprising an N-terminal signal sequence and the growth factor;
(b) growing a monocot plant from the transformed monocot plant cell for a time sufficient to produce seeds containing the growth factor; and
(c) harvesting the seeds from the plant.

2. The method of claim 1, wherein the promoter is from a monocot plant gene of a maturation-specific monocot plant storage protein or an aleurone- or embryo-specific monocot plant gene.

3. The method of claim 2, wherein the promoter is a member selected from the group consisting of rice glutelins, oryzins and prolamines, barley hordeins, wheat gliadins and glutenins, maize zeins and glutelins, oat glutelins, sorghum kafirins, millet pennisetins, rye secalins, lipid transfer protein Ltp1, chitinase Chi26 and Em protein Emp1.

4. The method of claim 1, wherein the promoter is derived from a cereal selected from the group consisting of rice, barley, wheat, oat, rye, corn, millet, triticale and sorghum.

5. The method of claim 1, wherein the promoter is selected from the group consisting of rice globulin Glb promoter and rice glutelin Gt1 promoter.

6. The method of claim 1, wherein the monocot plant seed-specific signal sequence is associated with a gene selected from the group consisting of glutelins, prolamines, hordeins, gliadins, glutenins, zeins, albumin, globulin, ADP glucose pyrophosphorylase, starch synthase, branching enzyme, Em, and lea.

7. The method of claim 1, wherein the monocot plant seed-specific signal sequence is associated with a gene selected from the group consisting of a-amylase, protease, carboxypeptidase, endoprotease, ribonuclease, DNase/RNAase, (1-3)-β-glucanase, (1-3)(1-4)-β-glucanase, esterase, acid phosphatase, pentosamine, endoxylanase, β-xylopyranosidase, arabinofuranosidase, β-glucosidase, (1-6)-β-glucanase, perioxidase, and lysophospholipase.

8. The method of claim 1, wherein the monocot plant seed-specific signal sequence is a rice glutelin Gt1 signal sequence.

9. The method of claim 1, wherein the monocot plant seed-specific signal sequence targets the polypeptide linked thereto to a subcellular compartment or tissue of a monocot plant seed endosperm cell.

10. The method of claim 9, wherein the subcellular compartment or tissue is selected from the group consisting of protein-storage body, vacuole, chloroplast, mitochondria and endoplasmic reticulum.

11. The method of claim 1, further comprising purifying the growth factor from the harvested seeds.

12. The method of claim 11, wherein said purifying step comprises at least one of the following steps:
(1) milling the harvested seeds to prepare a flour composition;
(2) preparing an extract of the harvested seeds; and
(3) preparing a protein fraction of the harvested seeds.

13. The method of claim 1, wherein the growth factor constitutes at least 0.1 weight percent of the total protein in the harvested seeds.

14. The method of claim 1, wherein the growth factor constitutes at least 0.25 weight percent of the total protein in the harvested seeds.

15. The method of claim 1, wherein the growth factor is selected from the group consisting of epidermal growth factor (EGF), a keratinocyte growth factor (KGF), an insulin-like growth factor (IGF), intestinal trefoil factor (ITF), a transforming growth factor (TGF), granulocyte colony-stimulating factor (GCSF), nerve growth factor (NGF) and a fibroblast growth factor (FGF).

16. The method of claim 1, wherein the growth factor produced in the method comprises one or more plant glycosyl groups.

17. A purified human growth factor obtained by the method of claim 1, wherein the growth factor comprises one or more plant glycosyl groups.

18. The human growth factor of claim 17, selected from the group consisting of epidermal growth factor (EGF), a keratinocyte growth factor (KGF), an insulin-like growth factor (IGF), intestinal trefoil factor (ITF), a transforming growth factor (TGF), granulocyte colony-stimulating factor (GCSF), nerve growth factor (NGF) and a fibroblast growth factor (FGF).

19. A transformed monocot plant cell, comprising
(i) a heterologous promoter from a monocot plant gene that has upregulated activity during seed maturation,
(ii) a first heterologous DNA sequence, operably linked to said promoter, encoding a monocot plant seed-specific signal sequence capable of targeting a polypeptide linked thereto to monocot plant seed endosperm, and
(iii) a second heterologous DNA sequence, linked in translation frame with the first DNA sequence, encoding a human growth factor, wherein the first DNA sequence and the second DNA sequence together encode a fusion protein comprising an N-terminal signal sequence and the growth factor.

20. The plant cell of claim 19, wherein the growth factor is selected from the group consisting of epidermal growth factor (EGF), a keratinocyte growth factor (KGF), an insulin-like growth factor (IGF), intestinal trefoil factor (ITF), a transforming growth factor (TGF), granulocyte colony-stimulating factor (GCSF), nerve growth factor (NGF) and a fibroblast growth factor (FGF).

21. A monocot plant seed product selected from the group consisting of whole seed, seed fraction, flour, extract, malt, protein fraction and purified protein, prepared from the harvested seeds obtained by the method of claim 1, wherein the growth factor constitutes at least 0.1 weight percent of the total protein in the harvested seeds.

22. The plant seed product of claim 21, wherein the growth factor constitutes at least 0.25 weight percent of the total protein in the harvested seeds.

23. The plant seed product of claim 21, wherein the growth factor is selected from the group consisting of epidermal growth factor (EGF), a keratinocyte growth factor (KGF), an insulin-like growth factor (IGF), intestinal trefoil factor (ITF), a transforming growth factor (TGF), granulocyte colony-stimulating factor (GCSF), nerve growth factor (NGF) and a fibroblast growth factor (FGF).

24. A vector, comprising
(i) a promoter from a monocot plant gene that has upregulated activity during seed maturation,
(ii) a first DNA sequence, operably linked to said promoter, encoding a monocot plant seed-specific signal sequence capable of targeting a polypeptide linked thereto to monocot plant seed endosperm, and
(iii) a second DNA sequence, linked in translation frame with the first DNA sequence, encoding a human growth factor, wherein the first DNA sequence and the second DNA sequence together encode a fusion protein comprising an N-terminal signal sequence and the growth factor.


SEQUENCE LISTING (28 sequences)

SEQ ID NO: 1 - DNA, 162 bp, Homo sapiens
  CDS (1)..(159)
aactccgactcggagtgccccctctcccacgacggttactgcctccacgacggggtctgcatgtacatcgaggccctcgacaagtacgcctgcaactgcgtcgtgggctacatcggcgagcggtgccagtaccgcgacctcaagtggtgggagctgcgctga

SEQ ID NO: 2 - DNA, 159 bp, Homo sapiens
aatggtgactctgaatgtcccctgtcccacgatgggtactgcctccatgatggtgtgtgcatgtatattgaagcattggacaagtatgcatgcaactgtgttgttggctacatcggggagcgatgtcagtaccgagacctgaagtggtgggaactgcgc

SEQ ID NO: 3 - DNA, 213 bp, Homo sapiens
  CDS (1)..(210)
ggcccagagaccctgtgcggtgcggagctggtggacgccctccagttcgtctgcggggaccggggcttctacttcaacaagccaacgggctacgggtcctcctcgcgccgcgccccccagaccggcatcgtggacgagtgctgcttccgctcctgcgacctccggcggctggagatgtactgcgccccactcaagcccgccaagagcgcctga

SEQ ID NO: 4 - DNA, 210 bp, Homo sapiens
ggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagct

SEQ ID NO: 5 - DNA, 183 bp, Homo sapiens
  CDS (1)..(180)
gaggagtacgtcgggctctccgctaaccaatgcgcggtcccggccaaggaccgggtggactgcggctacccccacgtgacgccgaaggagtgcaacaaccggggctgctgcttcgactcccgcatcccaggcgtgccgtggtgcttcaagcccctcacccgcaagacggagtgcacgttctga

SEQ ID NO: 6 - DNA, 183 bp, Homo sapiens
gaggagtacgtgggcctgtctgcaaaccagtgtgccgtgccggccaaggacagggtggactgcggctacccccatgtcacccccaaggagtgcaacaaccggggctgctgctttgactccaggatccctggagtgccttggtgtttcaagcccctgactaggaagacagaatgcaccttctga

SEQ ID NO: 7 - DNA, 786 bp, Oryza sativa
catgagtaatgtgtgagcattatgggaccacgaaataaaaagaacattttgatgagtcgtgtatcctcgatgagcctcaaaagttctctcaccccggataagaaacccttaagcaatgtgcaaagtttgcattctccactgacataatgcaaaataagatatcatcgatgacatagcaactcatgcatcatatcatgcctctctcaacctattcattcctactcatctacataagtatcttcagctaaatgttagaacataaacccataagtcacgtttgatgagtattaggcgtgacacatgacaaatcacagactcaagcaagataaagcaaaatgatgtgtacataaaactccagagctatatgtcatattgcaaaaagaggagagcttataagacaaggcatgactcacaaaaattcacttgcctttcgtgtcaaaaagaggagggctttacattatccatgtcatattgcaaaagaaagagagaaagaacaacacaatgctgcgtcaattatacatatctgtatgtccatcattattcatccacctttcgtgtaccacacttcatatatcataagagtcacttcacgtctggacattaacaaactctatcttaacatttagatgcaagagcctttatctcactataaatgcacgatgatttctcattgtttctcacaaaaagcggccgcttcattagtcctacaacaacatggcatccataaatcgccccatagttttcttcacagtttgcttgttcctcttgtgcgatggctccctagcc

SEQ ID NO: 8 - DNA, 1055 bp, Oryza sativa
ctgcagggaggagaggggagagatggtgagagaggaggaagaagaggaggggtgacaatgatatgtggggcatgtgggcacccaattttttaattcattcttttgttgaaactgacatgtgggtcccatgagatttattatttttcggatcgaatcgccacgtaagcgctacgtcaatgctacgtcagatgaagaccgagtcaaattagccacgtaagcgccacgtcagccaaaaccaccatccaaaccgccgagggacctcatctgcactggttttgatagttgagggacccgttgtatctggtttttcgattgaaggacgaaaatcaaatttgttgacaagttaagggaccttaaatgaacttattccatttcaaaatattctgtgagccatatataccgtgggcttccaatcctcctcaaattaaagggcctttttaaaatagataattgccttctttcagtcacccataaaagtacaaaactactaccaacaagcaacatgcgcagttacacacattttctgcacatttccgccacgtcacaaagagctaagagttatccctaggacaatctcattagtgtagatacatccattaatcttttatcagaggcaaacgtaaagccgctctttatgacaaaaataggtgacacaaaagtgttatctgccacatacataacttcagaaattacccaacaccaagagaaaaataaaaaaaaatctttttgcaagctccaaatcttggaaacctttttcactctttgcagcattgtactcttgctctttttccaaccgatccatgtcaccctcaagcttctacttgatctacacgaagctcaccgtgcacacaaccatggccacaaaaaccctataaaaccccatccgatcgccatcatctcatcatcagttcattaccaacaaacaaaagaggaaaaaaaacatatacacttctagtgattgtctgattgatcatcaatctagaggcggccgcatggctagcaaggtcgtcttcttcgcggcggcgctcatggcggccatggtggccatctccggc

SEQ ID NO: 9 - DNA, 976 bp, Triticum aestivum
ctgcaggccagggaaagacaatggacatgcaaagaggtaggggcagggaagaaacacttggagatcatagaagaacataagaggttaaacataggagggcataatggacaattaaatctacattaattgaactcatttgggaagtaaacaaaatccatattctggtgtaaatcaaactatttgacgcggatttactaagatcctatgttaattttagacatgactggccaaaggtttcagttagttcatttgtcacggaaaggtgttttcataagtccaaaactctaccaacttttttgcacgtcatagcatagatagatgttgtgagtcattggatagatattgtgagtcagcatggatttgtgttgcctggaaatccaactaaatgacaagcaacaaaacctgaaatgggctttaggagagatggtttatcaatttacatgttccatgcaggctaccttccactactcgacatggttagaagttttgagtgccgcatatttgcggaagcaatggcactactcgacatggttagaagttttgagtgccgcatatttgcggaagcaatggctaacagatacatattctgccaaaccccaagaaggataatcactcctcttagataaaaagaacagaccaatgtacaaacatccacacttctgcaaacaatacaccagaactaggattaagcccattacgtggctttagcagaccgtccaaaaatctgttttgcaagcaccaattgctccttacttatccagcttcttttgtgttggcaaactgcccttttccaaccgattttgtttcttctcacgctttcttcataggctaaactaacctcggcgtgcacacaaccatgtcctgaaccttcacctcgtccctataaaagcccatccaaccttacaatctcatcatcacccacaacaccgagcaccccaatctacagatcaattcactgacagttcactgatctaga

SEQ ID NO: 10 - DNA, 1009 bp, Oryza sativa
ctgcagtaatggatacctagtagcaagctagcttaaacaaatctaaattccaatctgttcgtaaacgttttctcgatcgcaattttgatcaaaactattgaaaacctcaattaaaccattcaaaatttttaatatacccaacaagagcgtccaaaccaaatatgtaaatatggatgtcatgataattgacttatgacaatgtgattatttcatcaagtctttaaatcattaattctagttgaaggtttatgttttcttatgctaaagggttatgtttatataagaatattaaagagcaaattgcaatagatcaacacaacaaatttgaatgtttccagatgtgtaaaaatatccaaattaattgttttaaaatagttttaagaaggatctgatatgcaagtttgatagttagtaaactgcaaaagggcttattacatggaaaattccttattgaatatgtttcattgactggtttattttacatgacaacaaagttactagtatgtcaataaaaaaatacaaggttacttgtcaattgtattgtgccaagtaaagatgacaacaaacatacaaatttatttgttcttttatagaaacacctaacttatcaaggatagttggccacgcaaaaatgacaacatactttacaattgtatcatcataaagatcttatcaagtataagaactttatggtgacataaaaaataatcacaagggcaagacacatactaaaagtatggacagaaatttcttaacaaactccatttgttttgtatccaaaagcataagaaatgagtcatggctgagtcatgatatgtagttcaatcttgcaaaattgcctttttgttaagtattgttttaacactacaagtcacatattgtctatacttgcaacaaacactattaccgtgtatcccaagtggccttttcattgctatataaactagcttgatcggtctttcaactcacatcaattagcttaagtttccattagcaactgctaatagct

SEQ ID NO: 11 - DNA, 839 bp, Oryza sativa
ctgcagtgtaagtgtagcttcttatagcttagtgctttactatcttcacaagcacatgctatagtattgttccaagatgaaagaataattcatccttgctaccaacttgcatgatattatatttgtgaatatcctatctcttggcttataatgaaatgtgctgctgggttattctgaccatggtatttgagagcctttgtatagctgaaaccaacgtatatcgagcatggaacagagaacaaaatgcaaggatttttttattctggttcatgccctggatgggttaatatcgtgatcatcaaaaaagatatgcataaaattaaagtaataaatttgctcataagaaaccaaaaccaaaagcacatatgtcctaaacaaactgcattttgtttgtcatgtagcaatacaagagataatatatgacgtggttatgacttattcactttttgtgactccaaaatgtagtaggtctaactgattgtttaaagtgatgtcttactgtagaagtttcatcccaaaagcaatcactaaagcaacacacacgtatagtccaccttcacgtaattctttgtggaagataacaagaaggctcactgaaaaataaaagcaaagaaaaggatatcaaacagaccattgtgcatcccattgatccttgtatgtctatttatctatcctccttttgtgtaccttacttctatctagtgagtcacttcatatgtggacattaacaaactctatcttaacatctagtcgatcactactttacttcactataaaaggaccaacatatatcatccatttctcacaaaagcattgagttcagtcccacaaaatctaga

SEQ ID NO: 12 - DNA, 1302 bp, Oryza sativa
ctgcagagatatggattttctaagattaattgattctctgtctaaagaaaaaaagtattattgaattaaatggaaaaagaaaaaggaaaaaggggatggcttctgctttttgggctgaaggcggcgtgtggccagcgtgctgcgtgcggacagcgagcgaacacacgacggagcagctacgacgaacgggggaccgagtggaccggacgaggatgtggcctaggacgagtgcacaaggctagtggactcggtccccgcgcggtatcccgagtggtccactgtctgcaaacacgattcacatagagcgggcagacgcgggagccgtcctaggtgcaccggaagcaaatccgtcgcctgggtggatttgagtgacacggcccacgtgtagcctcacagctctccgtggtcagatgtgtaaaattatcataatatgtgtttttcaaatagttaaataatatatataggcaagttatatgggtcaataagcagtaaaaaggcttatgacatggtaaaattacttacaccaatatgccttactgtctgatatattttacatgacaacaaagttacaagtacgtcatttaaaaatacaagttacttatcaattgtagtgtatcaagtaaatgacaacaaacctacaaatttgctattttgaaggaacacttaaaaaaatcaataggcaagttatatagtcaataaactgcaagaaggcttatgacatggaaaaattacatacaccaatatgctttattgtccggtatattttacaagacaacaaagttataagtatgtcatttaaaaatacaagttacttatcaattgtcaagtaaatgaaaacaaacctacaaatttgttattttgaaggaacacctaaattatcaaatatagcttgctacgcaaaatgacaacatgcttacaagttattatcatcttaaagttagactcatcttctcaagcataagagctttatggtgcaaaaacaaatataatgacaaggcaaagatacatacatattaagagtatggacagacatttctttaacaaactccatttgtattactccaaaagcaccagaagtttgtcatggctgagtcatgaaatgtatagttcaatcttgcaaagttgcctttccttttgtactgtgttttaacactacaagccatatattgtctgtacgtgcaacaaactatatcaccatgtatcccaagatgcttttttattgctatataaactagcttggtctgtctttgaactcacatcaattagcttaagtttccataagcaagtacaaatagctctaga

SEQ ID NO: 13 - DNA, 675 bp, Oryza sativa
ctgcagcatcggcttaggtgtagcaacacgactttattattattattattattattattattattttacaaaaatataaaatagatcagtccctcaccacaagtagagcaagttggtgagttattgtaaagttctacaaagctaatttaaaagttattgcattaacttatttcatattacaaacaagagtgtcaatggaacaatgaaaaccatatgacatactataattttgtttttattattgaaattatataattcaaagagaataaatccacatagccgtaaagttctacatgtggtgcattaccaaaatatatatagcttacaaaacatgacaagcttagtttgaaaaattgcaatccttatcacattgacacataaagtgagtgatgagtcataatattatttttcttgctacccatcatgtatatatgatagccacaaagttactttgatgatgatatcaaagaacatttttaggtgcacctaacagaatatccaaataatatgactcacttagatcataatagagcatcaagtaaaactaacactctaaagcaaccgatgggaaagcatctataaatagacaagcacaatgaaaatcctcatcatccttcaccacaattcaaatattatagttgaagcatagtagtagaatccaacaacaatctagag

SEQ ID NO: 14 - DNA, 1098 bp, Oryza sativa
ccaggcttcatcctaaccattacaggcaagatgttgtatgaagaagggcgaacatgcagattgttaaactgacacgtgatggacaagaatgaccgattggtgaccggtctgacaatggtcatgtcgtcagcagacagccatctcccacgtcgcgcctgcttccggtgaaagtggaggtaggtatgggccgtcccgtcagaaggtgattcggatggcagcgatacaaatctccgtccattaatgaagagaagtcaagttgaaagaaagggagggagagatggtgcatgtgggatccccttgggatataaaaggaggaccttgcccacttagaaaggagaggagaaagcaatcccagaagaatcgggggctgactggcactttgtagcttcttcatacgcgaatccaccaaaacacaggagtagggtattacgcttctcagcggcccgaacctgtatacatcgcccgtgtcttgtgtgtttccgctcttgcgaaccttccacagattgggagcttagaacctcacccagggcccccggccgaactggcaaaggggggcctgcgcggtctcccggtgaggagccccacgctccgtcagttctaaattacccgatgagaaagggaggggggggggggaaatctgccttgtttatttacgatccaacggatttggtcgacaccgatgaggtgtcttaccagttaccacgagctagattatagtactaattacttgaggattcggttcctaattttttacccgatcgacttcgccatggaaaattttttattcgggggagaatatccaccctgtttcgctcctaattaagataggaattgttacgattagcaacctaattcagatcagaattgttagttagcggcgttggatccctcacctcatcccatcccaattcccaaacccaaactcctcttccagtcgccgacccaaacacgcatccgccgcctataaatcccacccgcatcgagcctatcaagcccaaaaaaccacaaaccaaacgaagaaggaaaaaaaaaggaggaaaagaaaagaggaggaaagcgaagaggttggagagagacgctcgtctccacgtcgccgcc

SEQ ID NO: 15 - DNA, 432 bp, Hordeum vulgare
cttcgagtgcccgccgatttgccagcaatggctaacagacacatattctgccaaaaccccagaacaataatcacttctcgtagatgaagagaacagaccaagatacaaacgtccacgcttcagcaaacagtaccccagaactaggattaagccgattacgcggctttagcagaccgtccaaaaaaactgttttgcaaagctccaattcctccttgcttatccaatttcttttgtgttggcaaactgcacttgtccaaccgattttgttcttcccgtgtttcttcttaggctaactaacacagccgtgcacatagccatggtccggaatcttcacctcgtccctataaaagcccagccaatctccacaatctcatcatcaccgagaacaccgagaaccacaaaactagagatcaattcattgacagtccaccg

SEQ ID NO: 16 - DNA, 60 bp, Triticum aestivum
atggctaagcgcctggtcctctttgcggcagtagtcgtcgccctcgtggctctcaccgcc

SEQ ID NO: 17 - DNA, 72 bp, Oryza sativa
atggcaactaccattttctctcgtttttctatatacttttgtgctatgctattatgccagggttctatggcc

SEQ ID NO: 18 - DNA, 85 bp, Oryza sativa
atgtggacattaacaaactctatcttaacatctagtcgatcactactttacttcactataaaaggaccaacatatatcatccatt

SEQ ID NO: 19 - DNA, 72 bp, Oryza sativa
atggcgagttccgttttctctcggttttctatatacttttgtgttcttctattatgccatggttctatggcc

SEQ ID NO: 20 - DNA, 69 bp, Oryza sativa
atgaagatcattttcgtatttgctctccttgctattgttgcatgcaacgcttctgcacggtttgatgct

SEQ ID NO: 21 - DNA, 63 bp, Oryza sativa
atggccgcccgcgccgccgccgccgcgttcctgctgctgctcatcgtcgttggtcaccgcgcc

SEQ ID NO: 22 - DNA, 63 bp, Hordeum vulgare
atggctaagcggctggtcctctttgtggcggtaatcgtcgccctcgtggctctcaccaccgcc

SEQ ID NO: 23 - DNA, 1314 bp, Zea mays
atggagcacgtcatctcaatggaggagatcctcgggcccttctgggagctgctaccaccgccagcgccagagccagagcgagagcagcctccggtaaccggcatcgtcgtcggcagtgtcatagacgttgctgctgctggtcatggtgacggggacatgatggatcagcagcacgccacagagtggacctttgagaggttactagaagaggaggctctgacgacaagcacaccgccgccggtggtggtggtgccgaactcttgttgctcaggcgccctaaatgctgaccggccgccggtgatggaagaggcggtaactatggcgcctgcggcggtgagtagtgccgtagtaggtgaccccatggagtacaatgccatactgaggaggaagctggaggaggacctcgaggccttcaaaatgtggagggcggcctccagtgttgtgacctcagatcaacgttctcaaggctcaaacaatcacactggaggtagcagcatcaggaataatccagtgcagaacaagctgatgaacggcgaagatccaatcaacaataaccacgctcaaactgcaggccttggcgtgaggcttgctactagctcttcctcgagagatccttcaccatcagacgaagacatggacggagaagtagagattctggggttcaagatgcctaccgaggaaagagtgaggaaaagaaaggaatccaatagagaatcagccagacgctcgagatacaggaaagccgctcacctgaaagaactggaagaccaggtagcacagctaaaagccgagaattcttgcctgctgaggcgcattgccgctctgaaccagaagtacaacgacgctaacgtcgacaacagggtgctgagagcggacatggagaccctaagagctaaggtgaagatgggagaggactctctgaagcgggtgatagagatgagctcatcagtgccgtcgtccatgcccatctcggcgccgacccccagctccgacgctccagtgccgccgccgcctatccgagacagcatcgtcggctacttctccgccacagccgcagacgacgatgcttcggtcggcaacggtttcttgcgactgcaagctcatcaagagcctgcatccatggtcgtcggtggaactctgagcgccacagagatgaaccgagtagcagcagccacgcattgcgcgggggccatggagcacatccagacggcgatgggatccatgccgccgacctccgcctccggatctacaccgccgccgcaggattatgagctgctgggtccaaatggggccatacacatggacatgtattag

SEQ ID NO: 24 - DNA, 987 bp, Zea mays
atggacatgatctccggcagcactgcagcaacatcaacaccccacaacaaccaacaggcggtgatgttgtcatcccccattataaaggaggaagctagggacccaaagcagacacgagccatgccccaaataggtggcagtggggagcgtaagccgaggccgcaactacctgaggcgctcaagtgcccacgctgcgactccaacaacaccaagttttgctactacaacaattatagcatgtcacaaccacgctacttttgcaaggcttgccgccgctattggacacatggtggtaccctccgcaatgtccccattggtggtgggtgtcgcaagaacaaacatgcctctagatttgtcttgggctctcacacctcatcgtcctcatctgctacctatgcaccattatcccctagcaccaacgctagctctagcaatatgagcatcaacaaacatatgatgatggtgcctaacatgacgatgcctaccccaacgacaatgggcttattccctaatgtgctcccaacacttatgccgacaggtggaggcgggggctttgacttcactatggacaaccaacatagatcattgtccttcacaccaatgtctctacctagccaggggccagtgcctatgctggctgcaggagggagtgaggcaacaccgtctttcctagagatgctgagaggagggatttttcatggtagtagtagctataacacaagtctcacgatgagtggtggcaacaatggaatggacaagccattttcgctgccatcatatggtgcaatgtgcacaaatgggttgagtggctcaaccactaatgatgccagacaactggtggggcctcagcaggataacaaggccatcatgaagagcagtaataacaacaatggtgtatcattgttgaacctctactggaacaagcacaacaacaacaacaacaacaacaacaacaacaacaacaacaacaacaacaagggacaataa

SEQ ID NO: 25 - DNA, 3902 bp, Oryza sativa
atggagcgggtgttctccgtggaggagatctccgacccattctgggtcccgcctccgccgccgcagtcggcggcggcggcccagcagcagggcggcggcggcgtggcttcgggaggtggtggtggtgtagcggggggcggcggcggcgggaacgcgatgaaccggtgcccgtcggagtggtacttccagaagtttctggaggaggcggtgctcgatagccccgtcccgaaccctagcccgagggccgaagcgggagggatcaggggcgcaggaggggtggtgccggtcgatgttaagcagccgcagctctcggcggcggcgacgacgagcgcggtggtggaccccgtggagtacaacgcgatgctgaagcagaagctggagaaggacctcgccgcggtcgccatgtggagggtacagccattctccccccctctagtactcgagagcttactgagatcggcaatgctagctactgtttgcatcgaatgtttataggtatttagatcgggcatttctatagaccaatggcgtccatggtcttgcaatgcgctctgttgagtgtcggtggttggttcgactcatagtatgtagggttgtgcgtatgtacaaacggaagcttcatagacctcggtattgagattgcgatatcgatgcaacctgcgaattggcgatgtaatcagtcatattcttactaaactgcgagacagtggtttgtttgcaattgcaatatttttgtatggggctgcttaaactgtcattgcctttttagattggcaatatgtgactttatgcaagtatttgattgggcggatccaggaacaaaaagttggggggattcaacataccgagtacactggcataaacacatcatctcagtattaaactatgctaaaatgctattaagagacctttagcacctcttatcttatcaaccatggtgaaaaaattgaaggggggactcaggggggtatccatgggtccgatgggtgcaggggggactgagtcccccctgcacccacgttgaatccgccctggcatgcgtataagctgtcacagccatttctaggtgcttgtgcttagttgggtgatgtcagcttaatttgtcttttctatgtcgtcatcgattttctaagaaacgaaaaatagcctatttatgtgctccagaatttgatgatccctggcccttcatttgctgaaattagcctatttgttggttgcccttcagttttttcccagcttatgttgttgcaatgtgtggctatgcctcgttttgtgccctataatttattatttgcaattcatttttgtacatgacttaaaatgacactagagcaacatgcactgattggttatcctataatcatttatgtagttctgttcattttatcatgctagctcatgtcattttcatcttcaggcctctggcacagttccacctgagcgtcctggagctggttcatccttgctgaatgcagatgtttcacacataggcgctcctaattccatcggaggtacttatcttatctggttacattttcagattgttatgaaactacccaaatatcctgcacaattgcatgggattaaattttagtttctttgaaatagaagtagagttgtattgctgtcacgtcatcaaatagttctgaagctatgaataaataagttccgcatttgttagtgattctttgaacattagaattgttatgcttaagtagatagggttatgtttgtttggagttcccttaaatcatttcattgctgactgccagctggcaggagcatttgttgttgccttgaccatgaatgaagaccttcctgttctgagtgctcacaagaaaacatattttgattaatgcaccttgaatccttaggatcttgcaaagatgggcacttagctttagaattgagtagtacttaaatagctgttgttatcatgatttgtcctgtagtgaaatgtcgacaaaacaggaatgctacttttgacttctgatatttcatgcctggctttacttatgctctgtttggaacatgggcacatatcaggcaatgctactccagttcaaaacatgctaagtggcccaagtgggggatcgggctcacagttggtacagaatgttgatgtccttgtaaagcagcccaccagctcttcatcaagggagcagtcagatgatgatgacatgaagggagaagctgagaccactggaactgcaagacctgctgatcaaagattacaacgaaggtgatcattcattgcttccttgtaatatagattctgtacataattaacctacctcgtcatgcatgcatgtgtcctattttcaccttagccctttcagttggatttccactttcatccggtagcctttcagtttcctattgcatcgcatatatgatcttttacctaccatattagttctctgtgtgccatactcagtgcttagtgtctcgagcaagagaggaatttgtatggctattacacgtagcactttgctctctacttgtttattgacataagcaatttgggatgaattaaatctgagttcacatcatattccttatgtcacaagtttctgaaaccgattgtatctagtatctggttgatgcacccccatcttggatttgcaaatcaaagttatactccctagagagctttacctttcataaagcaattaccccaataaaccacggatttgatagctattgactatgattaccagaattcatttggcagctattttctcaatttaagtttggtattagtctcagttggctgtaaaataatgtcacggtagggtacatgtatgtgcagcatacaaggtatgggtgagttatgatatggacagtgtgtacaccccacatttgctcactaaaatcaaaatattcaaacgtcacgtgatgatatggtggattgcattataccttgtattgtttattatgttacttgtgctagacaataatataggctgttcttttgggtgattttgtatgaagatgttgagcaagcacttctcgatataatgctagttttgttgacctgttccaggaagcaatccaatcgggagtcagccaggcgctcaagaagcagaaaggcagctcacttgaatgagctggaggcacaggtgtgatagttcacatagttattttcgataagacataaaatcctaaattactggctactgacttcagttatggatttacttgttacaggtatcgcaattaagagtcgagaactcctcgctgttaaggcgtcttgctgatgttaaccagaagtacaatgatgctgctgttgacaatagagtgctaaaagcagatgttgagaccttgagagcaaaggtatgctatatatgccttttgcaatatgcatcccatggattgctactttggcttgtttcaaactttcaacgtgacttgtgtaccctgttattagaagaataatcccgcctaccattatactctataaatcaccatttggccagtccaaacatgattattaaatcaggtcaatctgaacattgaaatgtatcaaaaattcgcaggtgaagatggcagaggactcggtgaagcgggtgacaggcatgaacgcgttgtttcccgccgcttctgatatgtcatccctcagcatgccattcaacagctccccatctgaagcaacgtcagacgctgctgttcccatccaagatgacccgaacaattacttcgctactaacaacgacatcggaggtaacaacaactacatgcccgacataccttcttcggctcaggaggacgaggacttcgtcaatggcgctctggctgccggcaagattggccggccagcctcgctgcagcgggtggcgagcctggagcatctccagaagaggatgtgcggtgggccggcttcgtctgggtcgacgtcctga

SEQ ID NO: 26 - protein, 53 aa, Homo sapiens
NSDSECPLSHDGYCLHDGVCMYIEALDKYACNCVVGYIGERCQYRDLKWWELR

SEQ ID NO: 27 - protein, 70 aa, Homo sapiens
GPETLCGAELVDALQFVCGDRGFYFNKPTGYGSSSRRAPQTGIVDECCFRSCDLRRLEMYCAPLKPAKSA

SEQ ID NO: 28 - protein, 60 aa, Homo sapiens
EEYVGLSANQCAVPAKDRVDCGYPHVTPKECNNRGCCFDSRIPGVPWCFKPLTRKTECTF